(19) United States
(12) Patent Application Publication
(10) Pub. No.: US 2006/0258918 A1
(43) Pub. Date: Nov. 16, 2006

(54) Combination Analyte Measurement Device and Method of Use

(75) Inventors: John F. Burd, San Diego, CA (US); Paul Williams, San Diego, CA (US)

Correspondence Address: PROCOPIO, CORY, HARGREAVES & SAVITCH LLP, 530 B STREET, SUITE 2100, SAN DIEGO, CA, 92101, US

(73) Assignee: OCULIR, INC., San Diego, CA (US)

(21) Appl. No.: 11/460,173

(22) Filed: Jul. 26, 2006

Related U.S. Application Data
(63) Continuation-in-part of application No. 11/122,472, filed on May 5, 2005.
Application No. 11/122,472 is a continuation of application No. 10/824,214, filed on Apr. 14, 2004, now Pat. No. 6,975,892.

(51) Int. Cl. A61B 5/00

(52) U.S. Cl. 600/310; 600/316

(57) ABSTRACT

A combination non-invasive analyte reading and sample analyte reading device for both non-invasively measuring the presence, absence, or concentration of one or more analytes in a tissue of a subject and measuring the presence, absence, or concentration of one or more analytes in a biological sample of a subject includes a device housing; one or more outputs carried by the device housing; one or more inputs carried by the device housing to control operation of the device; a non-invasive analyte reader carried by the device housing and configured to measure the presence, absence, or concentration of one or more analytes in a tissue of a subject; and a sample analyte reader carried by the device housing and configured to measure the presence, absence, or concentration of one or more analytes in a biological sample of a subject.

### CROSS-REFERENCE TO RELATED APPLICATION

This application is a continuation-in-part application of U.S. patent application Ser. No. 11/122,472, filed May 5, 2005, which is a continuation application of U.S. patent application Ser. No. 10/824,214, filed Apr. 14, 2004, and claims the benefit of prior provisional application 60/513,396, filed on Oct. 21, 2003 under 35 U.S.C. 119(e). This application claims the benefit of these prior applications and these applications are incorporated by reference herein as though set forth in full.

### FIELD OF THE INVENTION

The present invention is related to non-invasive devices and methods to determine the presence, absence, or concentration of a wide range of analytes, such as, but not limited to, glucose, in the tissue of a subject.

### BACKGROUND OF THE INVENTION

Diabetes remains one of the most serious and under-treated diseases facing the worldwide healthcare system. Diabetes is a chronic disease where the body fails to maintain normal levels of glucose in the bloodstream. It is now the fifth leading cause of death from disease in the U.S. today and accounts for about 15% of the entire healthcare budget. People with diabetes are classified into two groups: Type 1 (formerly known as “juvenile onset” or “insulin dependent” diabetes, that are required to take insulin to maintain life) and Type 2 (formerly known as “adult onset” or “non-insulin dependent,” that may require insulin but may sometimes be treated by diet and oral hypoglycemic drugs). In both cases, without dedicated and regular blood glucose measurement, all patients face the possibility of the complications of diabetes that include cardiovascular disease, kidney failure, blindness, amputation of limbs and premature death.

The number of cases of diabetes in the U.S. has jumped 40% in the last decade. This high rate of growth is believed to be due to a combination of genetic and lifestyle origins that appear to be a long-term trend, including obesity and poor diet. The American Diabetes Association (ADA) and others estimate that about 17 million Americans and over 150 million people worldwide have diabetes, and it is estimated that up to 40% of these people are currently undiagnosed [American Diabetes Association, “Facts & Figures.”].

Diabetes must be “controlled” in order to delay the onset of the disease complications. Therefore, it is essential for people with diabetes to measure their blood glucose levels several times per day in an attempt to keep their glucose levels within the normal range (80 to 126 mg/dl). These glucose measurements are used to determine the amount of insulin or alternative treatments necessary to bring the glucose level to within target limits. Self-Monitoring of Blood Glucose (SMBG) is an ongoing process repeated multiple times per day for the rest of the patient's lifetime.

All currently FDA approved invasive or “less-invasive” (blood taken from the arm or other non-fingertip site) glucose monitoring products currently on the market require the drawing of blood in order to make a quantitative measurement of blood glucose. The ongoing and frequent measurement requirements (1 to possibly 10 times per day) presents all diabetic patients with pain, skin trauma, inconvenience, and infection risk resulting in a general reluctance to frequently perform the critical measurements necessary for selecting the appropriate insulin dose or other therapy.

These current product drawbacks have led to a poor rate of patient compliance. Among Type 1 diabetics, 39% measure their glucose levels less than once per day and 21% do not monitor their glucose at all. Among Type 2 diabetics who take insulin, only 26% monitor at least once per day and 47% do not monitor at all. Over 75% of non-insulin-taking Type 2 diabetics never monitor their glucose levels [Roper Starch Worldwide Survey]. Of 1,186 diabetics surveyed, 91% showed interest in a non-invasive glucose monitor [www.childrenwithdiabetes.com]. As such, there is both a tremendous interest and clinical need for a non-invasive glucose sensor.

The present invention seeks to supplement the currently used blood glucose measurement methods, devices and instruments, including invasive measures and the use of glucose test strips, with an optical non-invasive instrument.

Various methods have been developed related to non-invasive glucose sensing using a dermal testing site such as the finger or earlobe. These methods primarily employ instruments which measure blood glucose concentration by generating and measuring light only in the near-infrared radiation spectrum. For example, U.S. Pat. No. 4,882,492 (the '492 patent), expressly incorporated by reference herein, is directed to an instrument which transmits near-infrared radiation through a sample to be tested on the skin surface of a human. In the '492 patent, the near-infrared radiation that passes through the sample is split into two beams, wherein one beam is directed through a negative correlation filter and the second through a neutral density filter. The differential light intensity measured through the filters of the two light beams is proportional to glucose concentration according to the '492 patent.

U.S. Pat. No. 5,086,229 (the '229 patent), expressly incorporated by reference herein, is directed to an instrument which generates near-infrared radiation within the spectrum of about 600 to about 1100 nanometers. According to the '229 patent, a person places their finger in between the generated near-infrared radiation source and a detector, which correlates the blood glucose concentration based on the detected near-infrared radiation. Similarly, U.S. Pat. No. 5,321,265 (the '265 patent), expressly incorporated by reference herein, also measures the blood glucose level using both near-infrared radiation and the fingertip as a testing site. The detectors disclosed in the '265 patent further comprise silicon photocells and broad bandpass filters.

U.S. Pat. No. 5,361,758 (the '758 patent), expressly incorporated by reference herein, is directed to an instrument which measures near-infrared radiation that is either transmitted through or is reflected from the finger or earlobe of a human. In the '758 patent, the transmitted or reflected light is separated by a grating or prism, and the near-infrared radiation is detected and correlated with blood-glucose concentration. This instrument of the '758 patent also comprises an additional timing and control program wherein the device takes measurements specifically in between heartbeats and can also adjust for temperature.

U.S. Pat. No. 5,910,109 (the '109 patent), expressly incorporated by reference herein, is also directed to an instrument for measuring blood glucose concentration using near-infrared radiation and the earlobe as the testing site. The instrument of the '109 patent comprises four light sources of a very specific near-infrared emission spectrum, and four detectors having specific near-infrared detection spectra corresponding to the wavelength of the light sources. The signals detected by the four distinct detectors are averaged, and these averages are analyzed to determine blood glucose concentration according to the '109 patent.

The technique of using near-infrared radiation, wherein the near-infrared radiation is transmitted through or reflected from a dermal testing site and monitored for measuring glucose in vivo, is known to be inaccurate. The glucose concentration of interest is in the blood or the interstitial fluid, not on the surface of the dermis. Therefore these methods must penetrate down into the layers beneath the top layers of dermis. There are a number of substances in the dermis that can interfere with the near-infrared glucose signal. Additionally, there is a wide variation in the human dermis, both between individuals and within a given individual. Moreover, glucose simply lacks a satisfactory distinguishable “fingerprint” in the near-infrared radiation spectrum. Because near-infrared radiation is not sufficiently adsorbed by glucose and because of the level of tissue interferences found in the dermis, this technique is substantially less desirable for the accurate measurement of blood-glucose concentrations.

U.S. Pat. No. 6,362,144 (the '144 patent), expressly incorporated by reference herein, discloses using the fingertip as a testing site, however, the described instrument uses attenuated total reflection (ATR) infrared spectroscopy. According to the '144 patent, a selected skin surface, preferably the finger, is contacted with an ATR plate while ideally maintaining the pressure of contact. In the '144 patent, the skin is then irradiated with a mid-infrared beam, wherein the infrared radiation is detected and quantified to measure blood glucose levels. This technique is not ideal, however, if the surface of tissue from which the measurement is taken is very dense in the wavelength region of interest or is not amenable to direct contact with the ATR plate, such as an eye, conjunctiva, nose, mouth, or other orifice, cavity or piercing tract.

The minimal depth of peripheral capillaries in epithelial tissues is typically about 40 microns. Again, there are physical characteristics as well as a number of substances present in the skin that can interfere with the desired glucose-specific signal. While useful in the laboratory, both the near-infrared transmission methods and the ATR method mentioned above are not practical, or may not be adequate for use in monitoring blood glucose concentration in patients.

Methods have also been developed related to non-invasive glucose sensing using the eye as a testing site. For example, in both U.S. Pat. No. 3,958,560 (the '560 patent) and U.S. Pat. No. 4,014,321 (the '321 patent), both expressly incorporated by reference herein, a device utilizing the optical rotation of polarized light is described. In the '560 and the '321 patents, the light source and light detector are incorporated into a contact lens which is placed in contact with the surface of the eye whereby the eye is scanned using a dual source of polarized radiation, each source transmitting in a different absorption spectrum at one side of the cornea or aqueous humor. The optical rotation of the radiation that passes through the cornea correlates with the glucose concentration in the cornea according to the '560 and '321 patents. While this method would be termed, “non-invasive” because the withdrawal of blood is not required, it may still cause significant discomfort or distort vision of the user because of the need to place the sensor directly in contact with the eye.

U.S. Pat. No. 5,009,230 (the '230 patent), expressly incorporated by reference herein, uses a polarized light beam of near-infrared radiation within the range of 940 to 1000 nm. In the '230 patent, the amount of rotation imparted by glucose present in the bloodstream of the eye on the polarized light beam is measured to determine glucose concentration. Again, the accuracy is limited because glucose simply lacks a sufficiently distinguishable “fingerprint” in this near-infrared radiation spectrum.

Both U.S. Pat. No. 5,209,231 (the '231 patent), and International Publication No. WO 92/07511 (the '511 application), both expressly incorporated by reference herein, similarly disclose the use of polarized light, which is initially split by a beam splitter into a reference beam and a detector beam, and then transmitted through a specimen, preferably the aqueous humor of the eye. The amount of phase shift as compared between the transmitted reference and detector beams are correlated to determine glucose concentration in the '231 patent and '511 application. U.S. Pat. No. 5,535,743 (the '743 patent), expressly incorporated by reference herein, measures diffusely reflected light provided by the surface of the iris as opposed to the aqueous humor of the eye. According to the '743 patent, the measurement of optical absorption is possible whereas measurement of the optical rotation through the aqueous humor is not possible. In the '743 patent, the intensity of the diffusely reflected light, however, may be analyzed to obtain useful information on the optical properties of the aqueous humor, including blood-glucose concentration.

U.S. Pat. No. 5,687,721 (the '721 patent), expressly incorporated by reference herein, also discloses a method of measuring blood-glucose concentration by generating both a measurement and reference polarized light beam, and comparing the beams to determine the angle of rotation, which is attributable to the blood-glucose concentration. The preferable testing site disclosed, however, is the finger or other suitable appendage according to the '721 patent. The '721 patent further discloses and requires the use of a monochromatic laser and/or semi-conductor as a light source.

U.S. Pat. No. 5,788,632 (the '632 patent), expressly incorporated by reference herein, discloses a non-invasive instrument for determining blood-glucose concentration by transmitting a first beam of light through a first polarizer and a first retarder, then directing the light through the sample to be measured, transmitting the light through a second polarizer or retarder, and lastly detecting the light from the second detector. The rotation of measured polarized light is correlated to the blood-glucose concentration of the sample measured according to the '632 patent.

U.S. Pat. No. 5,433,197 (the '197 patent), expressly incorporated by reference herein, discloses a non-invasive instrument for determining blood glucose concentration using a broad-band of near-infrared radiation which illuminates the eye in such a manner that the energy passes through the aqueous humor in the anterior chamber of the eye and is then reflected from the iris. The reflected energy then passes back through the aqueous humor and the cornea and is collected for spectral analysis. According to the '197 patent, the electrical signals representative of the reflected energy are analyzed by univariate and/or multivariate signal processing techniques to correct for any errors in the glucose determination. Again, the accuracy of the instrument in the '197 patent is limited because glucose simply lacks a sufficiently distinguishable “fingerprint” in this near-infrared radiation spectrum.

Instruments and methods of using the body's naturally emitted radiation to measure blood-glucose concentration using the human body, and in particular, the tympanic membrane as a testing site have also been disclosed. U.S. Pat. Nos. 4,790,324; 4,797,840; 4,932,789; 5,024,533; 5,167,235; 5,169,235; and 5,178,464, expressly incorporated by reference herein, describe various designs, stabilization techniques and calibration techniques for tympanic non-contact thermometers. In addition, U.S. Pat. No. 5,666,956 (the '956 patent), expressly incorporated by reference herein, discloses an instrument which measures electromagnetic radiation from the tympanic membrane and computes monochromatic emissivity using Plank's law by measuring the radiation intensity, spectral distribution, and blackbody temperature. According to the '956 patent, the resultant monochromatic emissivity is variable depending on the spectral characteristics of the site measured, namely the blood-glucose concentration measured from the tympanic membrane. It should be noted, however, that the '956 patent equates skin surfaces of the body to a “gray-body” rather than a black-body with respect to its monochromatic emissivity. Therefore, according to the '956 patent, the accuracy of such skin surface-based methods utilizing natural black-body emitted radiation is not useful for analyte measurements, as compared to a method of subsurface analysis utilizing natural black-body radiation emitted from the tympanic membrane.

The human body naturally emits from its surfaces infrared radiation whose spectrum, or radiation signature, is modified by the presence, absence or concentration of analytes in the body tissues. The eye is particularly well suited as a testing site to detect this infrared radiation. For example, certain analytes, such as glucose, exhibit a minimal time delay in glucose concentration changes between the eye and the blood, and the eye provides a body surface with few interferences [Cameron et al., (3)2 DIABETES TECHNOL. THER., 202-207 (2001)]. There is, therefore, in the field of non-invasive blood analyte monitoring, an unmet need for a suitable instrument, and methodologies for using it, to accurately measure analyte concentrations, such as blood glucose concentration, as well as concentrations of other desired analytes, in subjects requiring this type of blood analyte measurement.

### SUMMARY OF THE INVENTION

Accordingly, an aspect of the invention involves a combination non-invasive analyte reading and sample analyte reading device for both non-invasively measuring the presence, absence, or concentration of one or more analytes in a tissue of a subject and measuring the presence, absence, or concentration of one or more analytes in a biological sample of a subject. The combination non-invasive analyte reading and sample analyte reading device includes a device housing; one or more outputs carried by the device housing; one or more inputs carried by the device housing to control operation of the device; a non-invasive analyte reader carried by the device housing and configured to measure the presence, absence, or concentration of one or more analytes in a tissue of a subject; and a sample analyte reader carried by the device housing and configured to measure the presence, absence, or concentration of one or more analytes in a biological sample of a subject.

Another aspect of the invention involves a method of using a combination non-invasive analyte reading and sample analyte reading device, the combination non-invasive analyte reading and sample analyte reading device including a device housing, one or more outputs carried by the device housing, one or more inputs carried by the device housing to control operation of the device, a non-invasive analyte reader carried by the device housing, and a sample analyte reader carried by the device housing. The method includes: a) using the sample analyte reader of the combination non-invasive analyte reading and sample analyte reading device to measure the presence, absence, or concentration of one or more analytes in a biological sample of a subject; b) using the non-invasive analyte reader of the combination non-invasive analyte reading and sample analyte reading device to non-invasively measure the presence, absence, or concentration of one or more analytes in a tissue of a subject; and c) outputting with the one or more outputs information related to the readings by the sample analyte reader and the non-invasive analyte reader.

A further aspect of the invention involves a method of using a combination non-invasive analyte reading and sample analyte reading device, the combination non-invasive analyte reading and sample analyte reading device including a device housing, one or more outputs carried by the device housing, one or more inputs carried by the device housing to control operation of the device, a non-invasive analyte reader carried by the device housing, and a sample analyte reader carried by the device housing. The method includes: a) using the sample analyte reader of the combination non-invasive analyte reading and sample analyte reading device to take a sample analyte measurement reading of the presence, absence, or concentration of one or more analytes in a biological sample of a subject; b) after step a, using the non-invasive analyte reader of the combination non-invasive analyte reading and sample analyte reading device to take a non-invasive measurement reading of the presence, absence, or concentration of one or more analytes in a tissue of a subject; c) comparing the non-invasive measurement reading with the sample analyte measurement reading to ensure that the non-invasive measurement reading conforms with the sample analyte measurement reading; and d) repeating steps b and c if the non-invasive measurement reading does not conform with the sample analyte measurement reading.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### BRIEF DESCRIPTION OF THE DRAWINGS

FIG. 1, Panel A provides a graphical illustration of the human eye. Panel B shows the high degree of vascularization in the conjunctiva, with veins (V) and arterioles (A).

FIG. 2 provides a graphical illustration of one embodiment of the present invention, wherein analyte concentration is measured from the mid-infrared radiation reflected back from the conjunctiva.

FIG. 3 provides a flowchart of one embodiment of the present invention, comprising a method wherein a remote access user can receive a subject's measured analyte concentrations which have been downloaded and stored in a computer system.

FIG. 4 provides a graph of multiple dose response measurements using detection of varying concentrations of glucose using polyethylene membranes as the measurement surface.

FIG. 5 shows a plot of the glucose concentration versus mid-infrared absorption using polyethylene membranes as the measurement surface.

FIG. 6 shows a plot of the results obtained from mid-infrared measurements of glucose concentration using a rabbit eye as the surface from which the measurements were made.

FIG. 7 shows a plot of human data obtained from the conjunctiva of the patient's eye measured using mid-infrared absorption to determine blood glucose concentration of the patient.

FIG. 8 shows a plot of the data obtained from a human diabetic patient in a glucose tracking study demonstrating a correlation of glucose concentration with mid-infrared absorption measured from the human eye surface.

FIG. 9 shows the correlation between glucose measurements taken from the conjunctiva according to the methods of the present invention (squares) and SMBG measurements (diamonds).

FIG. 10 shows a perspective view of an embodiment of a combination non-invasive analyte measurement and sample reading device.

FIG. 11 shows a block diagram of the non-invasive analyte measurement and sample reading device.

FIG. 12 shows a flow chart of an exemplary method of comparing a non-invasive analyte reading with respect to a sample analyte reading using the non-invasive analyte measurement and sample reading device illustrated in FIGS. 10 and 11.

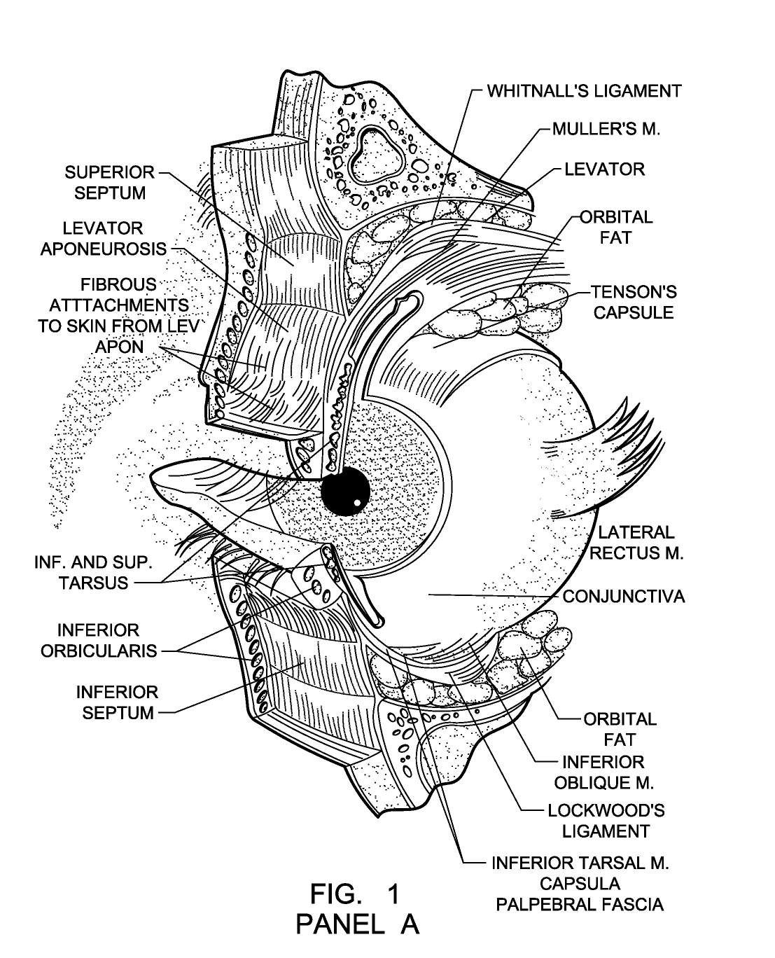

### DETAILED DESCRIPTION OF THE INVENTION

It is understood that the present invention is not limited to the particular methodologies, protocols, instruments, and systems, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include plural reference unless the context clearly dictates otherwise. Thus, for example, a reference to “a mid-infrared filter” is a reference to one or more filters and includes equivalents thereof known to those skilled in the art, and so forth. Further, for example, a reference to an instrument/monitor/reader for non-invasively measuring the presence, absence, or concentration of one or more analytes in an ocular element of a subject is a reference to the instrument/monitor/reader and includes devices (i.e., combination devices) that may integrate the instrument/monitor with one or more additional mechanisms. For example, but not by way of limitation, the instrument/monitor/reader may be integrated with a wireless communication device to wirelessly transmit/receive information and/or integrated with a sample analyte reader for reading a disposable sample collector (e.g., disposable chemical test strip, disposable printed circuit (PC) board).

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the present invention. All references cited herein are incorporated by reference herein in their entirety.

Definitions

Analyte: As used herein describes any particular substance or chemical constituent to be measured. Analyte may also include any substance in the tissue of a subject, in a biological fluid (for example, blood, interstitial fluid, cerebrospinal fluid, lymph fluid or urine), or is present in air that was in contact with or exhaled by a subject, which demonstrates an infrared radiation signature. Analyte may also include any substance which is foreign to or not normally present in the body of the subject. Analytes can include naturally occurring substances, artificial substances, metabolites, and/or reaction products. In some embodiments, the analyte for measurement by the devices and methods described herein is glucose. However, other analytes are contemplated as well, including, but not limited to, metabolic compounds or substances, carbohydrates such as sugars including glucose, proteins, glycated proteins, fructosamine, hemoglobin A1c, peptides, amino acids, fats, fatty acids, triglycerides, polysaccharides, alcohols including ethanol, toxins, hormones, vitamins, bacteria-related substances, fungus-related substances, virus-related substances, parasite-related substances, pharmaceutical or non-pharmaceutical compounds, substances, pro-drugs or drugs, and any precursor, metabolite, degradation product or surrogate marker of any of the foregoing. Other analytes are contemplated as well, including, but not limited, to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotimidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; nucleic acids (deoxyribonucleic acids and ribonucleic acids including native and variant sequences related to acetylator polymorphism, alcohol dehydrogenase, alpha 1-antitrypsin, cystic fibrosis, Down's syndrome, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, PKU, Plasmodium vivax, sexual differentiation, 21-hydroxylase); 21-deoxycortisol; desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosamimidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); neurotransmitters (such as glutamate, GABA, dopamine, serotonin), opioid neurotransmitters (such as endorphins, and dynorphins), neurokinins (such as substance P); succinylacetone; sulfadoxine; theophylline; thyrotropin (TSH); thyroxine (T4); thyroxine-binding globulin; trace elements; transferrin; UDP-galactose-4-epimerase; urea; prokaryotic and eukaryotic cell-surface antigens; peptidoglycans; lipopolysaccharide; uroporphyrinogen I synthase; vitamin A; white blood cells; and zinc protoporphyrin. Salts naturally occurring in blood or interstitial fluids can also constitute analytes in certain embodiments. The analyte can be naturally present in the biological fluid, for example, a metabolic product, an antigen, an antibody, and the like. Alternatively, the analyte can be introduced into the body, for example, a contrast agent for imaging, a radioisotope, a chemical agent, a fluorocarbon-based synthetic blood, or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); tricyclic antidepressants, benzodiazepines, acetaminophen (paracetamol, APAP), aspirin, methadone, hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (5HIAA).

Biological Sample: As used herein refers to blood, urine, tears, saliva, cerebrospinal fluid, lymph, tissue and other substances extractable from or secreted by the human body that include one or more analytes therein.

Conjunctiva: As used herein describes the membranous tissue that covers the exposed surface of the eye and the inner surface of the eyelids.

Electromagnetic Radiation: As used herein refers to any radiation energy, either generated from any source or naturally emitted, in the electromagnetic spectrum, namely, radiation energy having a frequency within the range of approximately 10²³ hertz to 0 hertz and a wavelength within the range of approximately 10⁻¹³ centimeter to infinity and including, in order of decreasing frequency, cosmic-ray photons, gamma rays, x-rays, ultraviolet radiation, visible light, infrared radiation, microwaves, and radio waves.

Far-Infrared Radiation: As used herein refers to any radiation, either generated from any source or naturally emitted, having wavelengths of about 50.00 to about 1000.00 microns.

Flooding: As used herein refers to broadly applying relatively widely diffused or spread-out rays of light onto a surface.

Focused: As used herein means mostly parallel rays of light that are caused to converge on a specific predetermined point.

Infrared Radiation: As used herein refers to any radiation, either generated from any source or naturally emitted, having wavelengths of about 0.78 to about 1000.00 microns.

Mid-Infrared Radiation: As used herein refers to any radiation, either generated from any source or naturally emitted, having wavelengths of about 2.50 microns to about 50.00 microns.

Mid-Infrared Radiation Detector: As used herein refers to any detector or sensor capable of registering infrared radiation. Examples of suitable infrared radiation detectors include, but are not limited to, a thermocouple, a thermistor, a microbolometer, and a liquid nitrogen cooled Mercury Cadmium Telluride (MCT) detector. The combined detected infrared radiation may be correlated with wavelengths corresponding to analyte concentrations using means such as the Fourier transform to produce high resolution spectra.

Near-Infrared Radiation: As used herein refers to any radiation, either generated or naturally emitted, having wavelengths of about 0.78 to about 2.50 microns.

Non-invasive: As used herein refers to a method or instrument that does not break a subject's skin nor any other tissue or surface barriers.

Ocular element: As used herein refers to an element of or relating to the eye such as, but not limited to the eyelid(s), epithelial cells, the aqueous humor, the vitreous humor, various layers of the cornea, iris, various layers of the sclera, conjunctiva, interstitial fluid in the conjunctiva, tears, the tear layer, and blood vessels.

Surface: As used herein refers to any part of a subject's body that may be exposed to the external environment, including, but not limited to, skin, the eye, ear, mouth, nose or any other orifice, body cavities, piercing tracts or other surface whether naturally occurring or artificial such as a surgically created surface.

Tears: The fluid secreted by the lacrimal gland and diffused between the eye and eyelids to moisten the parts and facilitate their motion.

Tear layer: The layer of fluid on the eye created by the tears.

Tissue: As used herein includes any tissue or component of a subject, including, but not limited to, skin, blood, body fluids, the eye, the tear layer of the eye, interstitial fluid, ocular fluid, bone, muscle, epithelium, fat, hair, fascia, organs, cartilage, tendons, ligaments, and any mucous membrane.

Non-Invasive Glucose Measurement

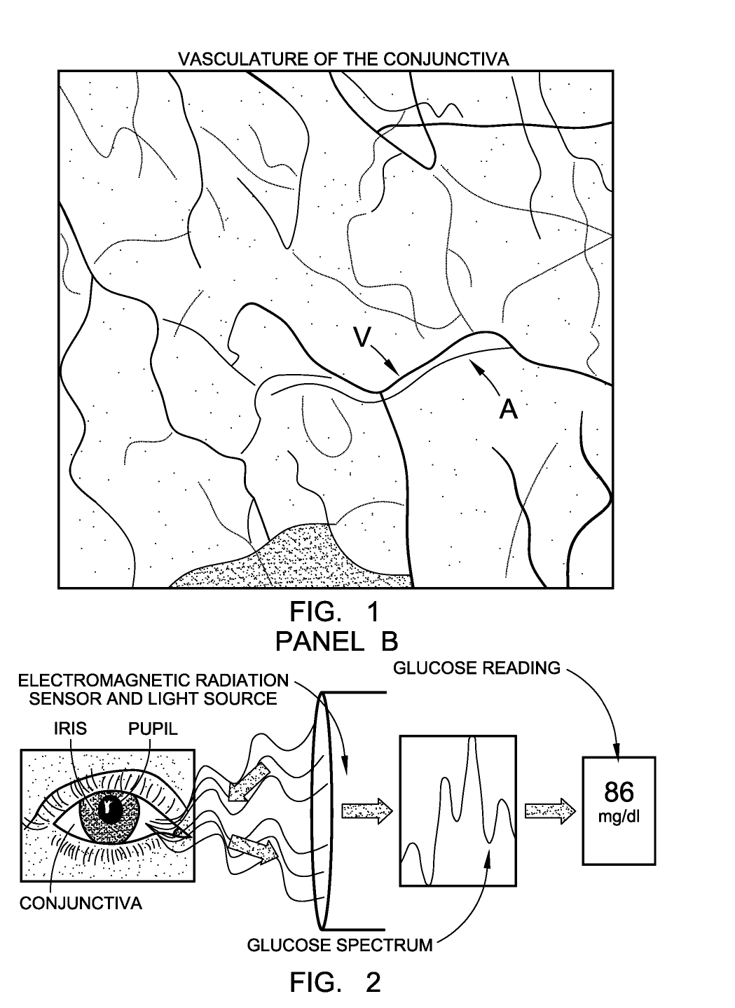

In one aspect of the present invention, electromagnetic radiation, and more preferably, infrared radiation, and even more preferably, mid-infrared radiation, is flooded onto the conjunctiva using a radiation source. This flooded mid-infrared radiation is reflected from the conjunctiva to a detector. The reflected radiation is detected by a mid-infrared detection instrument placed before the conjunctiva. The radiation signature of the reflected mid-infrared radiation is affected by the presence, absence, or concentration of one or more analytes. This provides a non-invasive method employing an instrument of the present invention to measure the presence, absence, or concentration of one or more analytes, such as, but not limited to, glucose, from a tissue such as, but not limited to, the conjunctiva of a subject (FIG. 2).

Although the conjunctiva is described as the ocular element for determining the presence, absence, or concentration of one or more analytes, in alternative embodiments, one or more other and/or additional ocular elements may be evaluated for determining the presence, absence, or concentration of one or more analytes. For example, ocular elements such as, but not by way of limitation, the epithelial cells, the aqueous humor, the vitreous humor, various layers of the cornea, iris, various layers of the sclera, conjunctiva, interstitial fluid in the conjunctiva, tears, the tear layer, and blood vessels may be evaluated by the devices and methods of the present invention.

Although the emitted and reflected electromagnetic radiation is frequently described herein as mid-infrared radiation, in alternative embodiments, other types of electromagnetic radiation are emitted and/or reflected.

There is substantial evidence that fluctuations in blood glucose levels are well correlated with glucose levels in the aqueous humor of the eye [Steffes, 1(2) DIABETES TECHNOL. THER., 129-133 (1999)]. In fact, it is estimated that the time delay between the blood and aqueous humor glucose concentration averages only about five minutes [Cameron et al., 3(2) DIABETES TECHNOL. THER., 201-207 (2001)]. The aqueous humor is a watery liquid that lies between the lens and cornea, which bathes and supplies the nutrients to the cornea, lens and iris. The glucose in the eye is located throughout the various components and compartments of the eye, including, but not limited to, epithelial cells, the aqueous humor, the vitreous humor, various layers of the cornea, iris, various layers of the sclera, conjunctiva, tears, the tear layer, and blood vessels. The eye, including, but not limited to, the tear layer and the conjunctiva, is both an ideal and suitable body surface for non-invasive measurement of the presence, absence or concentration of one or more analytes in the tissue of a subject. The conjunctiva is highly vascularized and generally consistent within an individual and between individuals, and provides ready access for the measurement of analytes. Therefore, the present invention is drawn to the use of the conjunctiva for analyte measurements that are non-invasive.

Measuring Infrared Radiation

When electromagnetic radiation is passed through a substance, it can either be absorbed or transmitted, depending upon its frequency and the structure of the molecules it encounters. Electromagnetic radiation is energy and hence when a molecule absorbs radiation it gains energy as it undergoes a quantum transition from one energy state (Eᵢₙᵢₜᵢₐₗ) to another (E_{final}). The frequency of the absorbed radiation is related to the energy of the transition by Planck's law: E_{final}−Eᵢₙᵢₜᵢₐₗ=E=hn=hc/l. Thus, if a transition exists which is related to the frequency of the incident radiation by Planck's constant, then the radiation can be absorbed. Conversely, if the frequency does not satisfy the Planck expression, then the radiation will be transmitted. A plot of the frequency of the incident radiation vs. some measure of the percent radiation absorbed by the sample is the radiation signature of the compound. The absorption of some amount of the radiation that is applied to a substance, or body surface containing substances, that absorbs radiation may result in a measurable decrease in the amount of radiation energy that actually passes through, or is affected by, the radiation absorbing substances. Such a decrease in the amount of radiation that passes through, or is affected by, the radiation absorbing substances may provide a measurable signal that may be utilized to measure the presence, absence or the concentration of one or more analytes.

One embodiment of the present invention provides a method for non-invasively measuring the blood-analyte concentration in a subject comprising the steps of generating electromagnetic radiation which is flooded onto the conjunctiva of the subject, detecting the reflected electromagnetic radiation, correlating the spectral characteristics of the detected electromagnetic radiation with a radiation signature that corresponds to the analyte concentration, and analyzing the detected electromagnetic radiation signature to give an analyte concentration measurement. In another embodiment, the method includes a filtering step before detection, by filtering the electromagnetic radiation reflected back from a body surface so that only wavelengths of about 8.00 microns to about 11.00 pass through the filter. In this embodiment, the filtering step may be accomplished using absorption filters, interference filters, monochromators, linear or circular variable filters, prisms or any other functional equivalent known in the art. The detecting step may be accomplished using any electromagnetic radiation sensor such as a thermocouple, thermistor, microbolometer, liquid nitrogen cooled MCT, or any other functional equivalent known in the art. In alternative embodiments, the detector includes specular reflection optics for surface reflective measurements, and diffuse reflection optics for deeper ocular element reflective measurements. Correlating the spectral characteristics of the detected electromagnetic radiation may comprise the use of a microprocessor to correlate the detected electromagnetic radiation signature with a radiation signature of an analyte. If the analyte being measured is glucose, then the radiation signature generated may be within the wavelength range within about 8.0 to about 11.0 microns. The analyzing step further comprises a microprocessor using algorithms based on Plank's law to correlate the absorption spectrum with a glucose concentration. In another embodiment of the present invention, the analyzing step may comprise the use of a transform, such as, but not limited to, Kramers-Kronig transform or other classical transform known in the art, to transform the detected radiation signature to the analyte spectra for correlation.

In another embodiment of the present invention, where glucose is the analyte of interest, an instrument comprising an electromagnetic radiation detector and a display may be held up to the conjunctiva of a subject. The electromagnetic radiation from the conjunctiva may optionally be filtered so that only wavelengths of about 8.0 microns to about 11.0 microns reach the electromagnetic radiation detector. The radiation signature of the electromagnetic radiation detected by the detector may then be correlated with a radiation signature that corresponds to a glucose concentration. The radiation signature may then be analyzed to give an accurate glucose concentration measurement. The measured glucose concentration may be displayed.

In another embodiment of the present invention, an instrument comprising an electromagnetic radiation generator, an electromagnetic radiation detector and a display may be held up to the conjunctiva of a subject. Electromagnetic radiation may be generated by the instrument and used for flooding or alternatively aiming a focused beam onto the conjunctiva of a subject. The electromagnetic radiation generated may be broad band or narrow band radiation, and may also be filtered to allow only desired wavelengths of radiation to reach the body surface. Any analyte, such as glucose, present in any constituent of the conjunctiva may absorb some of the generated radiation. The electromagnetic radiation that is not absorbed may be reflected back to the instrument. The reflected electromagnetic radiation may optionally be filtered so that only wavelengths of about 8.0 microns to about 11.0 microns reach electromagnetic radiation detector. The radiation signature of the electromagnetic radiation detected by the detector may then be correlated with a radiation signature that corresponds to analyte, such as glucose, concentration. The radiation signature may be analyzed to give an analyte, such as glucose, concentration. The measured analyte, such as glucose, concentration may be displayed by the instrument.

Infrared radiation may be generated by the instrument of the present invention. Such infrared radiation may be generated by any suitable generator including, but not limited to, a narrow band wavelength generator or a broadband wavelength generator. In one embodiment of the present invention, an instrument may comprise a mid-infrared radiation generator. In another embodiment of the present invention, the instrument comprises a light source with one or more filters to restrict the wavelengths of the light reaching the conjunctiva. The mid-infrared generator may further comprise a heating element. The heating element of this embodiment may be a Nernst glower (zirconium oxide/yttrium oxides), a NiChrome wire (nickel-chromium wire), and a Globar (silicon-carbon rod), narrow band or broad band light emitting diodes, or any other functional equivalent known in the art. Mid-infrared radiation has wavelengths in the range of about 2.5 microns to about 50.0 microns. Analytes typically have a characteristic “fingerprint” or “signature” or “radiation signature” with respect to their mid-infrared radiation spectrum that results from the analyte's affect on the mid-infrared radiation, such as absorption. Glucose in particular has a distinct spectral “fingerprint” or “signature” in the mid-infrared radiation spectrum, at wavelengths between about 8.0 microns to about 11.0 microns. This radiation signature of glucose may be readily generated for a wide variety of glucose concentrations utilizing a wide variety of body surfaces, such as the conjunctiva, for taking radiation signature data. In one embodiment of the present invention, an instrument may comprise a mid-infrared radiation filter, for filtering out all mid-infrared radiation not within a range of wavelengths from about 8.0 to about 11.0 microns. In other embodiments the filter is selected to filter out all mid-infrared radiation other than other than the wavelengths that provide the radiation signature of the desired analyte, such as glucose. Filtering mid-infrared radiation may be accomplished using absorption filters, interference filters, monochromators, linear or circular variable filters, prisms or any other functional equivalent known in the art.

In one embodiment of the present invention, the instrument may also comprise a mid-infrared radiation detector for detecting mid-infrared radiation. The mid-infrared radiation detector can measure the naturally emitted or reflected mid-infrared radiation in any form, including in the form of heat energy. Detecting the naturally emitted or reflected mid-infrared radiation may be accomplished using thermocouples, thermistors, microbolometers, liquid nitrogen cooled MCT, or any other functional equivalent known in the art. Both thermocouples and thermistors are well known in the art and are commercially available. For example, thermocouples are commonly used temperature sensors because they are relatively inexpensive, interchangeable, have standard connectors and can measure a wide range of temperatures [http://www.picotech.com]. In addition, Thermometrics' product portfolio comprises a wide range of thermistors (thermally sensitive resistors) which have, according to type, a negative (NTC), or positive (PTC) resistance/temperature coefficient [http://www.thermometrics.com].

The instrument of the present invention may also comprise a microprocessor. The microprocessor of this embodiment correlates the detected electromagnetic radiation with a radiation signature whose spectral characteristics provide information to the microprocessor about the analyte concentration being measured. The microprocessor of this embodiment analyzes the resultant radiation signature using suitable algorithms such as those based on Plank's law, to translate the radiation signature into an accurate analyte concentration measurement in the sample being measured.

It is readily apparent to those skilled in the art that a broad band light source may be modulated by an interferometer, such as in Fourier transform spectroscopy, or by an electro-optical or moving mask, as in Hadamard transform spectroscopy, to encode wavelength information in the time domain. A discrete wavelength band may be selected and scanned in center wavelength using, for example, an acousto-optical tuned filter. The instrument of the present invention having a radiation source, comprises one or more electromagnetic radiation sources, which provide radiation at many wavelengths, and also comprises one or more electromagnetic radiation detectors. The instrument may further comprise one or more filter or wavelength selector to remove, distinguish or select radiation of a desired wavelength, before or after detection by the detector.

Clinical Applications

It may be required for diabetes patients and subjects at risk for diabetes to measure their blood glucose levels regularly in an attempt to keep their blood glucose levels within an acceptable range, and to make an accurate recordation of blood-glucose levels for both personal and medical records. In one aspect of the present invention, the instrument may also comprise an alphanumeric display for displaying the measured blood-glucose concentration. The alphanumeric display of this embodiment may comprise a visual display and an audio display. The visual display may be a liquid crystal display (LCD), a plasma display panel (PDP), and a field emission display (FED) or any other functional equivalent known in the art. An audio display, capable of transmitting alphanumeric data and converting this alphanumeric data to an audio display, may be provided with an audio source comprising recorded audio clips, speech synthesizers and voice emulation algorithms or any other functional equivalent known in the art.

Self-Monitoring of Blood Glucose (SMBG) is an ongoing process repeated multiple times per day for the rest of the diabetic patient's lifetime. Accurate recordation of these measurements are crucial for diagnostic purposes. A facile storage and access system for this data is also contemplated in this invention. In one aspect of the present invention, an instrument for non-invasively measuring blood-glucose concentration further comprises a microprocessor and a memory which is operatively linked to the microprocessor for storing the blood glucose measurements. The instrument of this embodiment further comprises a communications interface adapted to transmit data from the instrument to a computer system. In this embodiment the communications interface selected may include, for example, serial, parallel, universal serial bus (USB), FireWire, Ethernet, fiber optic, co-axial, twisted pair cables, a wireless communication link (e.g., WLAN, WIFI, Bluetooth, infrared) or any other functional equivalent known in the art.

In addition to storing blood-glucose measurement data within an instrument, the present invention includes a computer system for downloading and storing these measurement data to facilitate storage and access to this information. The present invention further contemplates a computer processor, a memory which is operatively linked to the computer processor, a communications interface adapted to receive and send data within the computer processor, and a computer program stored in the memory which executes in the computer processor. The computer program of this embodiment further comprises a database, wherein data received by the database may be sorted into predetermined fields, and the database may be capable of graphical representations of the downloaded analyte concentrations. The graphical representations of this embodiment may include, but are not limited to, column, line, bar, pie, XY scatter, area, radar, and surface representations.

The computer system contemplated by the present invention should be accessible to a remote access user via an analogous communications interface for use as a diagnostic, research, or other medically related tool. Physicians, for example, could logon to the computer system via their analogous communications interface and upload a patient's blood-glucose measurements over any period of time. This information could provide a physician with an accurate record to use as a patient monitoring or diagnostic tool such as, for example, adjusting medication levels or recommending dietary changes. Other remote access users contemplated may include research institutes, clinical trial centers, specialists, nurses, hospice service providers, insurance carriers, and any other health care provider.

The present invention has demonstrated that glucose can be non-invasively measured using a mid-infrared signal from an ocular element. Studies have been performed in a variety of systems, in vitro studies using glucose solutions in a gelatin matrix, and human studies including a diabetic human volunteer with varying blood glucose concentrations.

All studies, including the human studies, clearly demonstrate the dose-response of blood glucose concentrations using mid-infrared measurement techniques compared to standard SMBG monitoring test strips.

### EXAMPLES

The following examples are provided to describe and illustrate the present invention. As such, they should not be construed to limit the scope of the invention. Those in the art will well appreciate that many other embodiments also fall within the scope of the invention, as it is described hereinabove and in the claims.

### Example 1

Experimental In-Vitro Model to Test Precision and Accuracy of the Instrument

Instrumentation

The instrument used for the mid-infrared measurements was the SOC 400 portable FTIR. The SOC 400 portable FTIR is based on an interferometer and was originally designed for the U.S. Army to detect battlefield gases. This instrument has been modified to allow measurements on in vitro models using glucose solutions in a gelatin matrix and also on human eyes. These modifications have included the installation of a filter to allow only energy in the 7 to 13 micron region to be measured and also the modification of the faceplate to permit easier placement of the instrument for human studies.

In Vitro Studies

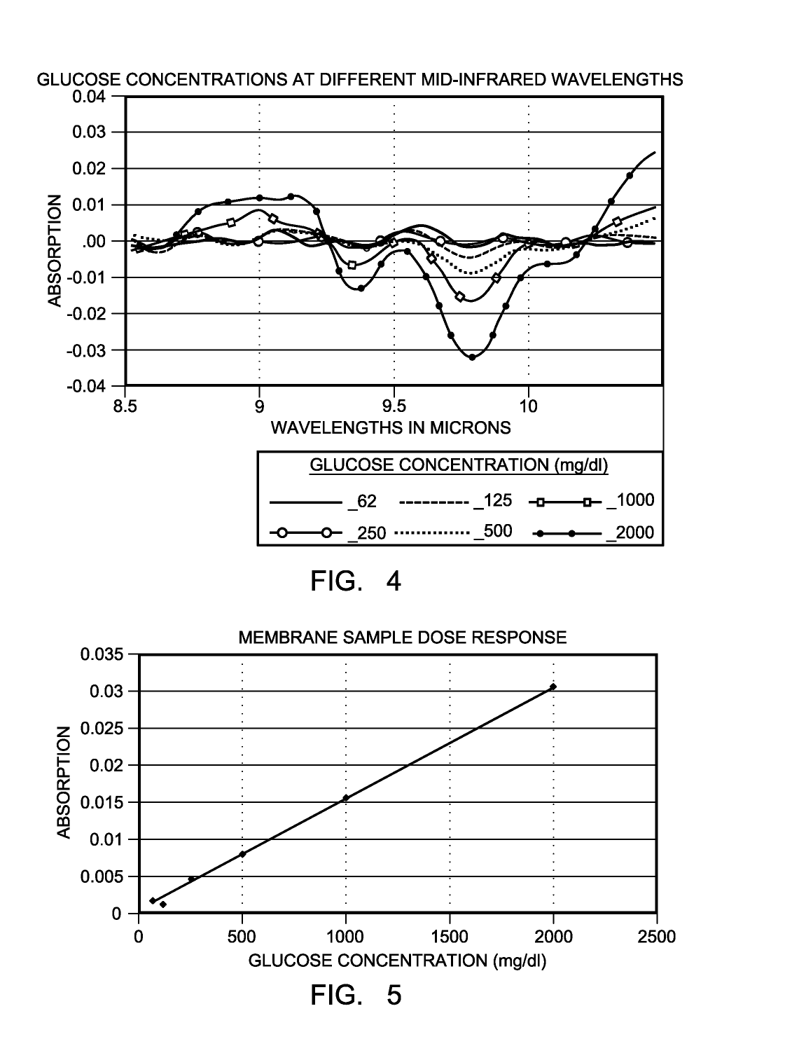

Studies were performed to demonstrate that solutions with varying concentrations of glucose would give a mid-infrared dose-response. Hydrophilic polyethylene membranes from Millipore Corporation were saturated with glucose solutions with concentrations at 2000 mg/dl and lower. The series of curves generated in this experiment are shown in FIG. 4. For this plot, the following equation was used: Absorption=−ln (sample spectrum/gold reference spectrum). When the glucose concentration is plotted against the absorption at 9.75 microns, the plot shown in FIG. 5 was observed. These studies confirmed that glucose concentration can be measured in an aqueous environment in the mid-infrared wavelength range.

### Example 2

Experimental Rabbit Model

Ketamine Anesthetized Rabbit studies

As noted in the scientific literature (Cameron et al., DIABETES TECH. THER., (1999) 1(2): 135-143), rabbits anesthetized with Ketamine experience a rapid and marked increase in blood glucose concentration, due to the release of glucose from the liver. We have confirmed this in a series of experiments and observed that the rabbit blood sugar can change from ˜125 mg/dl to ˜325 mg/dl in 60 minutes, as measured with a LXN ExpressView blood glucose meter. These experiments require a preliminary use of gas anesthesia (Isoflorane) prior to the use of Ketamine. The rabbit was immobilized such that after anesthesia, the eyeball was available for measurements with the SOC 400 portable FTIR. Once the animal was unconscious, a drop of blood from a vein was taken and tested on a blood glucose test strip with the LXN ExpressView blood glucose meter. Such samples were taken every fifteen minutes throughout the study. The gas must be discontinued in order for the Ketamine effect to fully manifest itself. The drying out of the eye may be prevented by suturing the eyelids and using the sutures to open the eye for the measurement and then allowing them to close after the measurement to moisten the eyeball.

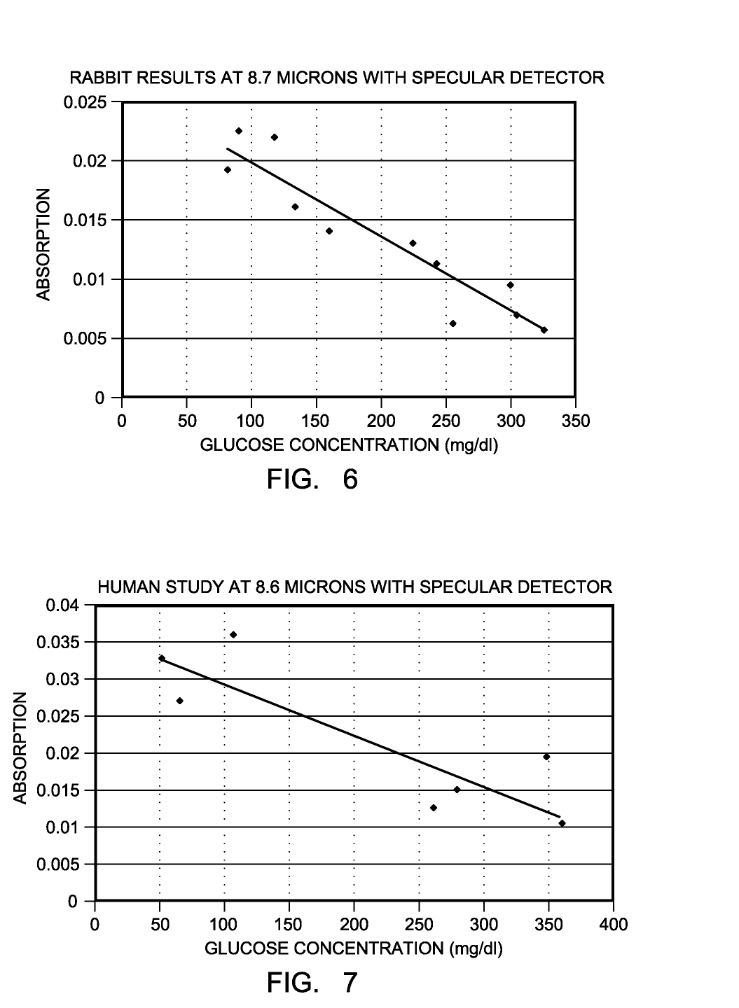

The data from the rabbit study measuring glucose concentration from an ocular element yielded the results with a regression coefficient (R squared) of 0.86, shown in FIG. 6.

### Example 3

Human Clinical Study

Human Studies

Several studies were performed with non-diabetic and diabetic human volunteers. Prior to performing these studies it was confirmed that the infrared radiation being used poses no health hazard.

Several experiments with a diabetic volunteer were performed. The subject was asked to adjust his food intake and insulin administration in order to have his glucose levels move from approximately 100 to 300 mg/dl over a three to four hour timeframe. During the study, the patient took duplicate fingerstick glucose measurements and was scanned with the SOC 400 approximately every five minutes. Prior to collecting the infrared scan, the instrument operator aligned the SOC 400 with the subjects' eye to attempt to collect the strongest signal being reflected off of the eye.

In one study performed on the patient using the SOC 400 measuring off of the surface of the patient's eyeball, the following correlation was observed, as shown in FIG. 7. As seen, the correlation of the signal with the glucose concentration is clear.

Human Study using the SOC 400

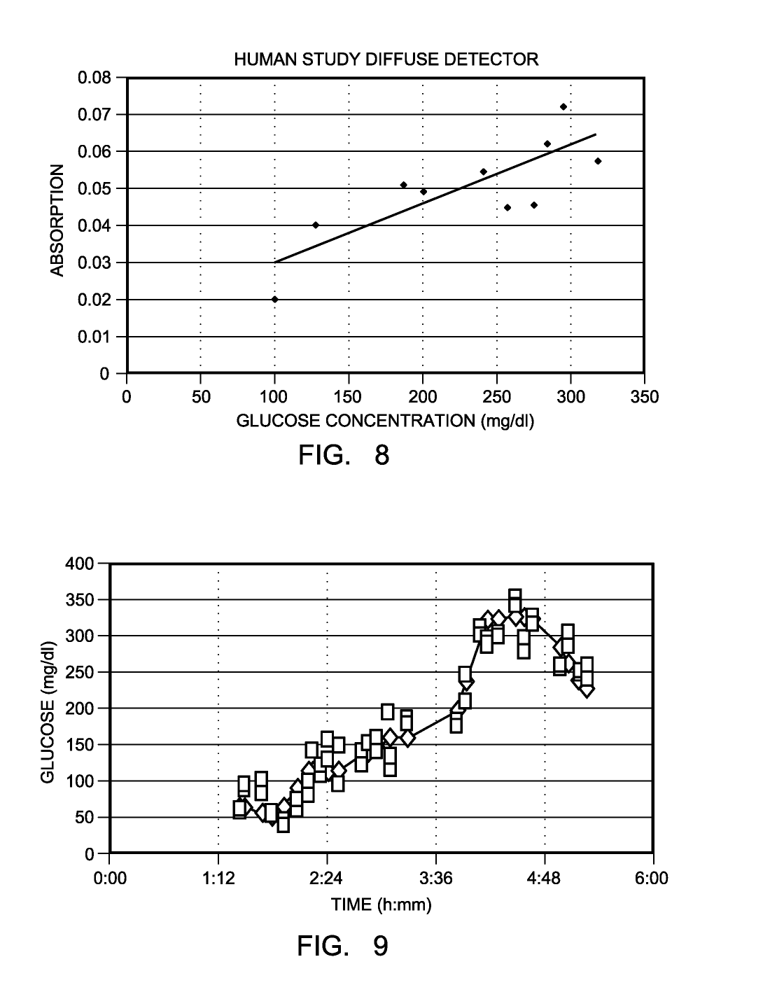

A glucose tracking study was performed using the diffuse detector for the SOC 400. A glucose tracking study was performed with a diabetic volunteer and the results shown in FIG. 8 demonstrate that the glucose concentration changes were clearly detected and measured using an instrument and method of the present invention. The correlation between the measurements taken with the instrument of the present invention using the methods of the present invention is shown in FIG. 9. Measurements using the instruments and methods of the present invention showed very close correlation to SMBG measurements (squares and diamonds respectively).

### Example 4

A Method Wherein a Remote Access User Can Receive a Subject's Measured Analyte Concentrations That Have Been Downloaded and Stored in a Computer System

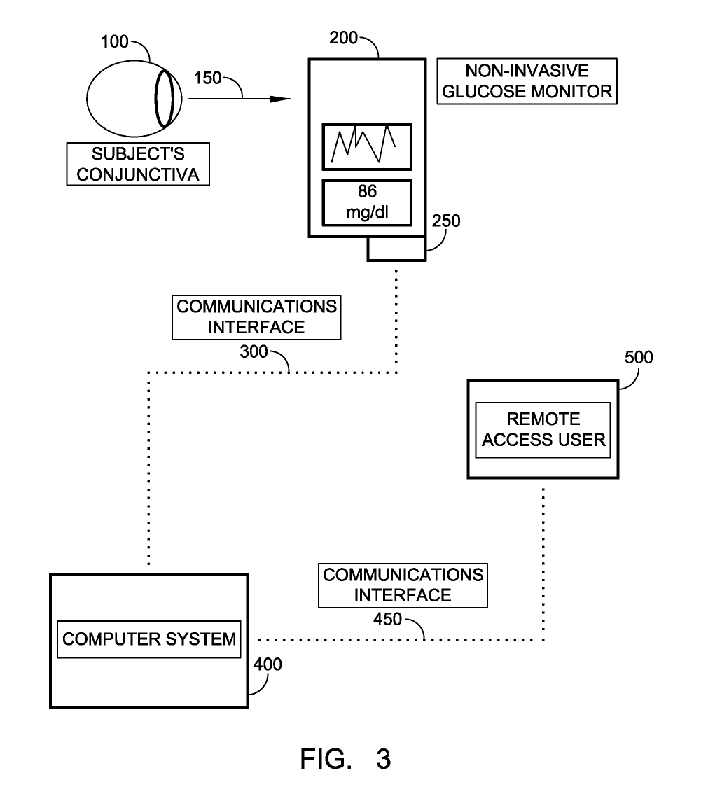

One aspect of the present invention relates to a method of downloading and storing a subject's measured analyte concentrations (FIG. 3). A subject first measures the analyte concentration from a tissue such as their eye (100), whereby reflected electromagnetic radiation (150) is measured using a non-invasive instrument (200). The non-invasive instrument (200) further comprises a communications interface (250) which is capable of connecting (300) the non-invasive instrument (200) through the communications interface (250) to a computer system (400). The communications interface (250) is specifically adapted to transmit data from the instrument to the computer system (400). The computer system (400) comprises a computer processor, a computer program which executes in the computer processor, and an analogous communications interface (450). The measured analyte concentrations from the non-invasive instrument (200) are downloaded via the communications interface (250) to the computer system (400). A remote access user (500), having a computer system with an analogous communications interface (450) is capable of retrieving the downloaded measured analyte concentrations from the computer system (400). The communications interfaces (250, 450) may include, for example, serial, parallel, universal serial bus (USB), FireWire, Ethernet, fiber optic, co-axial, twisted pair cables, and/or a wireless communication link (e.g., WLAN, WIFI, Bluetooth, infrared). This information is used, for example, to provide data, warnings, advice or assistance to the patient or physician, and to track a patient's progress throughout the course of the disease.

### Example 5

A Combination Non-Invasive Analyte Measurement And Sample Reading Device And Methods Of Using The Same

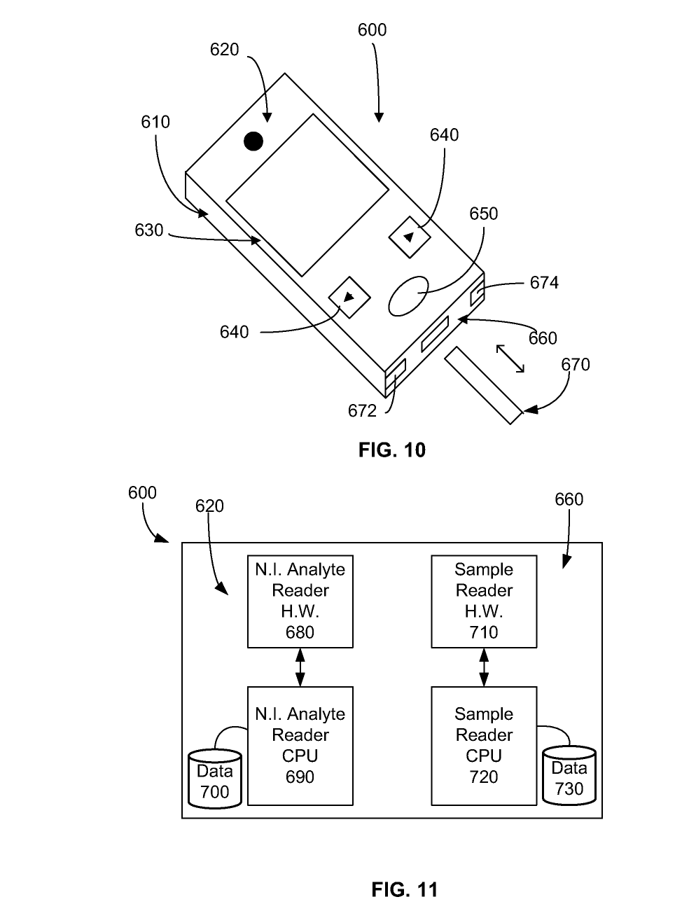

With reference to FIGS. 10-12, another aspect of the present invention relates to a combination non-invasive analyte measurement and sample reading device (“device”) 600 and methods of using the same.

The combination device 600 includes two analyte readers: 1) a non-invasive analyte reader 620, and 2) a sample analyte reader 660.

The non-invasive analyte reader 620 will be described below in conjunction with an optical non-invasive method to detect the presence, absence, or concentration of glucose in the conjunctiva of a subject by utilizing emitted or reflected mid-infrared radiation from the tissue in order to make the glucose measurements. However, in alternative embodiments, the non-invasive analyte reader 620 is used to detect the presence, absence, or concentration of one or more analytes in the tissue of a subject by utilizing emitted or reflected electromagnetic radiation from the tissue in order to make the analyte measurements. In such alternative embodiments, the non-invasive method involves the eye and/or other tissues; the analyte is glucose and/or other analytes; and/or the electromagnetic radiation is near-infrared, mid-infrared, far-infrared and/or other types of electromagnetic energy.

The sample analyte reader 660 will be described below as either an electrochemical reader for measuring an electrical response from a drop of blood on a disposable printed circuit board to detect blood sugar/glucose level or an optical chemical strip reader for measuring a change in color from a drop of blood on a chemical test strip to detect blood sugar level. However, in alternative embodiments, the sample analyte reader 660 is used to detect the presence, absence, or concentration of one or more analytes in a biological sample from a subject. In such alternative embodiments, the one or more analytes include glucose and/or other analytes; the biological sample includes blood and/or other biological samples; and/or the sample analyte reader includes an electrochemical reader, an optical chemical strip reader, and/or other type of sample analyte readers.

FIG. 10 shows a perspective view of an embodiment of the device 600. The device 600 includes a housing 610 that houses suitable electronics, hardware, and software to perform the functions of the device 600 described herein, a non-invasive (“N.I.”) analyte reader 620, a display screen 630, toggle keys 640, function key 650, a sample analyte reader 660 for reading a disposable sample collector 670, a disposable sample collector holder 672, and a lance holder 674.

The toggle keys 640 are used to toggle through menu options or other information on the display screen 630 and/or memory 700 of the device 600. The function key 650 is used to select highlighted information on the display screen 630, and may be used to affect other functions such as, but not by way of limitation, initiating a reading and turning the device 600 on/off.

In an alternative embodiment, one or more outputs (e.g., speaker, vibration mechanism, alarm) in addition to, or as an alternative to, the display screen 630 are used for outputting information related to the readings by the sample analyte reader 660 and the non-invasive analyte reader 620.

The disposable sample collector holder 672 includes a cover that is displaceable for accessing the holder 672. The holder 672 is used to conveniently store disposable sample collectors 670 (e.g., disposable chemical test strips, disposable printed circuit (PC) boards).

Similarly, the lance holder 674 includes a cover that is displaceable for accessing the holder 674. The holder 674 is used to conveniently store disposable lances for penetrating the subject's skin to obtain a blood sample.

With reference additionally to FIG. 11, the noninvasive reader 620 includes non-invasive analyte reader hardware 680, a non-invasive analyte reader processing unit 690, and memory 700, which may include software executable by the processor 690, the software having one or more modules for performing the functions described herein. In the embodiment shown, the non-invasive analyte reader hardware 680 includes an electromagnetic radiation generator and an electromagnetic radiation detector. In an alternative embodiment, where naturally emitted electromagnetic radiation is detected, the non-invasive analyte reader hardware 680 includes an electromagnetic radiation detector, but not an electromagnetic radiation generator. As shown in FIG. 2, the device 600 is held adjacent to the user's eye so that the non-invasive reader 620 is directed at the user's eye. Electromagnetic radiation is generated by the electromagnetic radiation generator and is used for flooding (or alternatively aiming a focused beam) onto an ocular element, for example, the conjunctiva of a subject. In alternative embodiments, the electromagnetic radiation generated is broad band or narrow band radiation, and/or is filtered to allow only desired wavelengths of radiation to reach the conjunctiva. One or more analytes, such as, but not limited to, glucose, present in any constituent of the conjunctiva absorbs some of the generated radiation. The electromagnetic radiation that is not absorbed is reflected back to the electromagnetic radiation detector. As indicated above, in an embodiment of the invention, the reflected electromagnetic radiation is filtered so that only certain, select wavelengths reach the electromagnetic radiation detector. The radiation signature of the electromagnetic radiation detected by the detector is then correlated by the non-invasive analyte reader processing unit 690 with a radiation signature (e.g., in memory 700) that corresponds to blood sugar level or glucose concentration. The radiation signature is analyzed by the non-invasive analyte reader processing unit 690 to give blood sugar level. The blood sugar level is displayed in the display screen 630. In alternative embodiments, the noninvasive reader 620 determines the presence, absence, or concentration of one or more analytes in addition to or other than glucose. In alternative embodiments, the noninvasive reader 620 determines the presence, absence, or concentration of one or more analytes from tissue other than the conjunctiva. In alternative embodiments, the noninvasive reader 620 is a different type of non-invasive reader for determining the presence, absence, or concentration of one or more analytes from tissue other than that described above.

With reference additionally to FIG. 11, two embodiments of the sample analyte reader 660 will be described.

In a first embodiment of the sample analyte reader 660, the sample analyte reader 660 is an electrochemical reader and the disposable sample collector 670 is a disposable printed circuit (PC) board. The sample reader hardware 710 includes the appropriate socket/recess, mechanical mechanisms and electronics for reading the disposable PC board once inserted into the socket/recess of the electrochemical reader 660. The user's finger or other site (e.g., upper arm, forearm, base of thumb and thigh) is washed with soap and water, and/or cleaned with an alcohol swab. A blood sample is drawn from the subject's finger (or other site) using a lance. A drop of the blood sample is placed on the disposable PC board 670. The disposable PC board 670 is inserted into the socket/recess of the electrochemical reader 660. The electrochemical reader 660 measures an electrical response from the drop of blood on the disposable printed circuit board 670 to detect blood sugar level. The measured electrical response is correlated by the sample reader processing unit 720 with an electrical signature (e.g., in memory 730) that corresponds to the appropriate blood sugar level. The measured electrical response is analyzed by the sample reader processing unit 720 to determine blood sugar level. The blood sugar level is displayed in the display screen 630. The PC board 670 is disposed of after use.

In a second embodiment of the sample analyte reader 660, the sample analyte reader 660 is an optical chemical strip reader and the disposable sample collector 670 is a chemical test strip. The sample reader hardware 710 includes the appropriate socket/recess, mechanical mechanisms, optics, and electronics for optically reading the chemical test strip 670 once inserted into the socket/recess of the optical chemical strip reader 660. The user's finger or other site (e.g., upper arm, forearm, base of thumb and thigh) is washed with soap and water, and/or cleaned with an alcohol swab. A blood sample is drawn from the subject's finger (or other site) using a lance. A drop of the blood sample is placed on the chemical test strip 670. The sample end of the chemical test strip 670 is inserted into the socket/recess of the optical chemical strip reader 660. The optical chemical strip reader 660 measures a change in color from a drop of blood on the chemical test strip 670 to detect blood sugar level. The color read by the optical chemical strip reader 660 is correlated by the sample reader processing unit 720 with information representative of a color (e.g., in memory 730) that corresponds to the appropriate blood sugar level. The measured color is analyzed by the sample reader processing unit 720 to determine blood sugar level. The blood sugar level is displayed in the display screen 630. The chemical test strip 670 is disposed of after use.

In alternative embodiments, the sample analyte reader 660 is used to detect the presence, absence, or concentration of one or more analytes in a biological sample from a subject. In such alternative embodiments, the one or more analytes include glucose and/or other analytes; the biological sample includes blood and/or other biological samples; and/or the sample analyte reader includes an electrochemical reader, an optical chemical strip reader, and/or other types of sample analyte readers.

It should be noted that in the embodiment shown in FIG. 11, the device includes dual processors, a non-invasive analyte reader processing unit 690 and a sample reader processing unit 720, which are isolated from each other (e.g., on separate ASICs). This isolates the readers 620, 660 so that, for example, software upgrades/modifications for one of the readers 620, 660 does not change or affect the operation of the other reader 620, 660. Isolation of the readers 620, 660 can be advantageous if one of the readers 620, 660 qualifies as an adjunct device by an appropriate administration agency. In alternative embodiments, the processing units 690, 720 are not isolated from each other (e.g., they communicate with each other or the functions are carried out by a single processing unit).

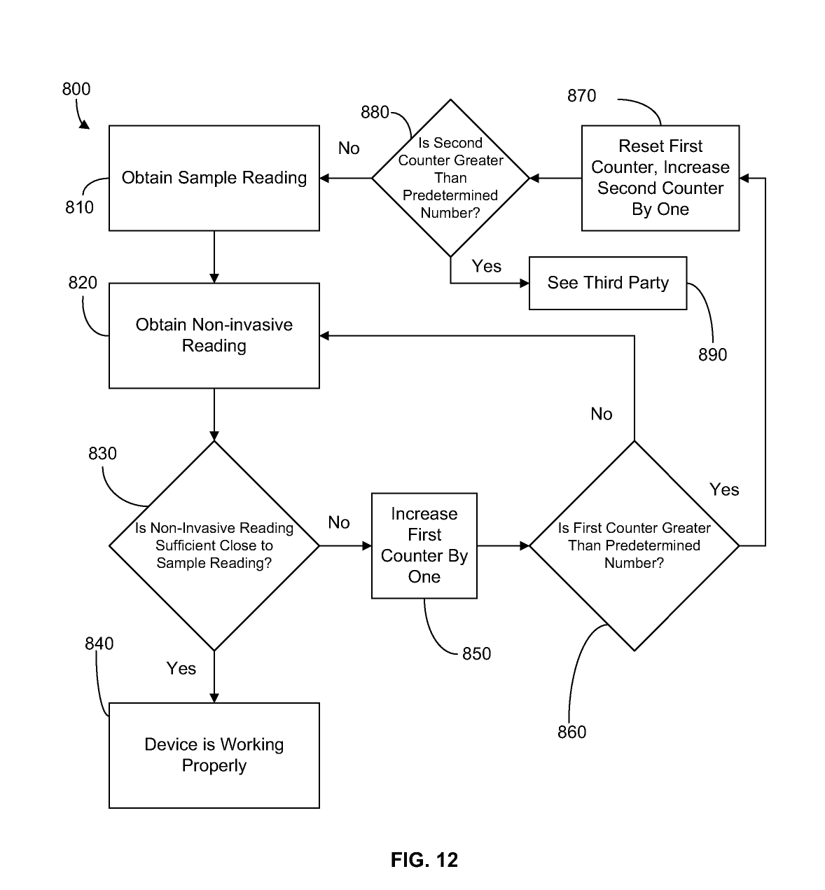

With reference to FIG. 12, an exemplary method 800 of comparing a reading taken by the non-invasive analyte reader 620 with respect to a reading taken by the sample analyte reader 660 will be described. To ensure that the non-invasive reader 620 is providing accurate, consistent readings relative to the sample reader 660, the readings taken by the non-invasive reader 620 are compared relative to the readings taken by the sample reader 660. One or more modules residing in memory 700, memory 730, and/or other memory of the device 600 include the instructions for performing the comparing process described herein.

First, at step 810, a sample reading is taken by the sample reader 660. The user's finger or other site (e.g., upper arm, forearm, base of thumb and thigh) is washed with soap and water, and/or cleaned with an alcohol swab. A blood sample is drawn from the subject's finger (or other site) using a lance. A drop of the blood sample is placed on the disposable PC board 670. The disposable PC board 670 is inserted into the socket/recess of the electrochemical reader 660. The electrochemical reader 660 measures an electrical response from the drop of blood on the disposable printed circuit board 670 to detect blood sugar level. The sample reading is stored in memory 730.

Alternatively, if the sample analyte reader 660 is an optical chemical strip reader and the disposable sample collector 670 is a chemical test strip, the same initial steps described above are completed and a drop of the blood sample is placed on the chemical test strip 670. The sample end of the chemical test strip is inserted into the socket/recess of the optical chemical strip reader 660. The optical chemical strip reader 660 measures a change in color from a drop of blood on the chemical test strip 670 to detect blood sugar level. The sample reading is stored in memory 730.

Next, at step 820, a non-invasive reading is taken by the non-invasive reader 620. As shown in FIG. 2, the device 600 is held adjacent to the user's eye so that the non-invasive reader is directed at the user's eye. Electromagnetic radiation generated by the electromagnetic radiation generator is flooded (or alternatively a focused beam is aimed) onto the conjunctiva of the subject. Glucose present in any constituent of the conjunctiva absorbs some of the generated radiation. The electromagnetic radiation that is not absorbed is reflected back to the electromagnetic radiation detector. Alternatively, in the above-described embodiment where the non-invasive analyte reader hardware does not include an electromagnetic radiation generator, naturally emitted electromagnetic energy is emitted from the subject's eye. The radiation signature of the electromagnetic radiation detected by the detector is then correlated by the non-invasive analyte reader processing unit 690 with a radiation signature that corresponds to blood sugar level or glucose concentration, and the blood sugar level is displayed in the display screen 630. The non-invasive reading is stored in memory 700.

Next, at step 830, a comparison is made between the stored non-invasive reading and sample analyte reading and a determination is made as to whether the non-invasive reading is sufficiently close or conforms to the sample reading to ensure that the non-invasive reader 620 is providing accurate blood sugar level readings and is working properly. In an alternative embodiment, prior to the comparing and determining step, a translation step occurs to translate the units from the sample reading and/or the non-invasive reading into the same units. As an example, but not by way of limitation, step 830 may involve a determination as to whether the non-invasive blood sugar level reading is within 2% of the sample blood sugar level reading. If the non-invasive blood sugar level reading is sufficiently close to the sample blood sugar level reading, control passes on to step 840, where the comparison process is considered complete and the device 600 is considered working properly. As described in more detail further below, in an alternative embodiment of the invention, at step 840, the non-invasive analyte reader 620 and/or the sample analyte reader 660 is calibrated/re-calibrated based on the readings taken by the non-invasive analyte reader 620 and the sample analyte reader 660 to improve accuracy and precision. If the non-invasive blood sugar level reading is not sufficiently close to the sample blood sugar level reading, control passes on to step 850, where a first counter is increased by an increment of one and a determination is made at step 860 as to whether the first counter is greater than a predetermined number (e.g., 2, 3, 4, etc.). If the first counter is not greater than the predetermined number, then the subject is prompted to obtain another non-invasive reading at step 820. If the first counter is greater than the predetermined number, then control is passed on to step 870, where the first counter is reset to an initial reading and a second counter is increased by one. Control is passed on to step 880, where a determination is made as to whether the second counter is greater than a predetermined number (e.g., 2, 3, 4, etc.). If the second counter is not greater than the predetermined number, then the subject is prompted to obtain another sample reading at step 810. In alternative embodiments, the subject is prompted to take another sample reading immediately after the prior sample reading occurred or the device 600 allows the subject to take another sample reading at the next regularly scheduled time (e.g., breakfast, lunch, dinner, bedtime, night) and the comparison process is continued at that later time. If the second counter is greater than the predetermined number, then control is passed on to step 890, where the subject is instructed to see a third party (e.g., contact customer assistance). In an alternative embodiment, step 890 includes automatically disabling the non-invasive reader 620, which may be later reset with proper authorization. In a further embodiment, as described in more detail further below, at step 890, the non-invasive analyte reader 620 and/or the sample analyte reader 660 is calibrated/re-calibrated based on the readings taken by the non-invasive analyte reader 620 and the sample analyte reader 660 to improve accuracy and precision.

In alternative embodiments, one or more of steps 810-830 are completed multiple times (e.g., 2 times, 3 times, 4 times, 5 times, etc.) even if the non-invasive blood sugar level reading is sufficiently close to the sample blood sugar level reading at step 830 before the comparison process is completed and the device 600 is considered to be working properly and/or calibrated/re-calibrated.

In an alternative embodiment, the user is prompted to take multiple non-invasive readings (i.e., step 820 is repeated multiple times), which are averaged, and the average is compared to the sample reading in step 830.

In alternative embodiments, one or more of steps 810-830 are completed multiple times (e.g., 2 times, 3 times, 4 times, 5 times, etc.) even if the non-invasive blood sugar level reading is sufficiently close to the sample blood sugar level reading at step 830 before the comparison process is completed and the device 600 is considered to be working properly and/or calibrated/recalibrated. In such embodiments, the subject is prompted to take another sample reading immediately after the prior sample reading occurred or the device 600 allows the subject to take another sample reading at the next regularly scheduled time (e.g., breakfast, lunch, dinner, bedtime, night) and the comparison process is continued at that later time. In further embodiments, the device 600 includes a timer or counter to have the device 600 to perform the comparison process 800 periodically, after a predetermined number of hours of using the device 600 (e.g., every 50 hours, 100 hours, 500 hours, 1000 hours, etc) and/or after a predetermined number of uses (e.g., every 50 uses, 100 uses, 500 uses, 1000 uses, etc). In still further embodiments, the device 600 averages the non-invasive blood sugar level reading and the sample blood sugar level reading to obtain an average blood sugar level reading or the device 600 performs a different mathematical function on the non-invasive blood sugar level reading and the sample blood sugar level reading to obtain a result related to the non-invasive blood sugar level reading and the sample blood sugar level reading.

In another embodiment of the invention, the non-invasive analyte reader 620 is calibrated/re-calibrated based on the one or more readings taken by the non-invasive analyte reader 620 and the sample analyte reader 660. For example, but not by way of limitation, after one or more non-invasive analyte readings and sample analyte readings taken in the method 800 above, the device 600 may determine that the non-invasive analyte reader 620 is consistently reading blood sugar level 5 mg/dL too low. The device 600 may then be calibrated/re-calibrated based on this information so that further non-invasive analyte readings with the non-invasive analyte reader 620 are more accurate and precise.

Similarly, in a further embodiment of the invention, the sample analyte reader 660 is calibrated/re-calibrated based on the one or more readings taken by the sample analyte reader 660 and the non-invasive analyte reader 620. For example, but not by way of limitation, after one or more non-invasive analyte readings and sample analyte readings taken in the method 800 above, the device 600 may determine that the sample analyte reader 660 is consistently reading blood sugar level 5 mg/dL too low. The device 600 may then be calibrated/re-calibrated based on this information so that further sample analyte readings with the sample analyte reader 660 are more accurate and precise.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

What is claimed is:

1. A combination non-invasive analyte reading and sample analyte reading device for both non-invasively measuring the presence, absence, or concentration of one or more analytes in a tissue of a subject and measuring the presence, absence, or concentration of one or more analytes in a biological sample of a subject, comprising:
a. a device housing;
b. one or more outputs carried by the device housing;
c. one or more inputs carried by the device housing to control operation of the device;
d. a non-invasive analyte reader carried by the device housing and configured to measure the presence, absence, or concentration of one or more analytes in a tissue of a subject; and
e. a sample analyte reader carried by the device housing and configured to measure the presence, absence, or concentration of one or more analytes in a biological sample of a subject.

2. The combination non-invasive analyte reading and sample analyte reading device of claim 1, wherein the non-invasive analyte reader includes:
a. a radiation source configured to expose at least a portion of the tissue of the subject to electromagnetic radiation;
b. a radiation detector configured to obtain a measurement of electromagnetic radiation from the tissue; and
c. a microprocessor for determining the presence, absence, or concentration of one or more analytes by determining a radiation signature of the electromagnetic radiation, and correlating the radiation signature of the electromagnetic radiation with information representative of the presence, absence, or concentration of one or more analytes.

3. The combination non-invasive analyte reading and sample analyte reading device of claim 1, wherein the sample analyte reader is an electrochemical reader for measuring an electrical response from a biological sample on a disposable printed circuit board to measure the presence, absence, or concentration of one or more analytes in the biological sample.

4. The combination non-invasive analyte reading and sample analyte reading device of claim 1, wherein the sample analyte reader is an optical chemical strip reader for measuring a change in color from a biological sample on a chemical test strip to measure the presence, absence, or concentration of one or more analytes in the biological sample.

5. The combination non-invasive analyte reading and sample analyte reading device of claim 1, wherein the non-invasive analyte reader and the sample analyte reader have separate respective microprocessors so that the functions of the non-invasive analyte reader and the sample analyte reader are isolated.

6. The combination non-invasive analyte reading and sample analyte reading device of claim 1, wherein the combination non-invasive analyte reading and sample analyte reading device includes one or more modules configured to compare one or more readings taken by the non-invasive analyte reader with one or more readings taken by the sample analyte reader to ensure that the one or more non-invasive measurement readings conform with the one or more sample analyte measurement readings.

7. A method of using a combination non-invasive analyte reading and sample analyte reading device, the combination non-invasive analyte reading and sample analyte reading device including a device housing, one or more outputs carried by the device housing, one or more inputs carried by the device housing to control operation of the device, a non-invasive analyte reader carried by the device housing, and a sample analyte reader carried by the device housing, comprising:
a) using the sample analyte reader of the combination non-invasive analyte reading and sample analyte reading device to measure the presence, absence, or concentration of one or more analytes in a biological sample of a subject;
b) using the non-invasive analyte reader of the combination non-invasive analyte reading and sample analyte reading device to non-invasively measure the presence, absence, or concentration of one or more analytes in a tissue of a subject;
c) outputting with the one or more outputs information related to the readings by the sample analyte reader and the non-invasive analyte reader.

8. The method of claim 7, wherein the non-invasive analyte reader includes a radiation source, a radiation detector, and a microprocessor, and using the non-invasive analyte reader of the combination non-invasive analyte reading and sample analyte reading device includes exposing at least a portion of the tissue of the subject to electromagnetic radiation from the radiation source; obtaining a measurement of electromagnetic radiation from the tissue using the radiation detector, and using the microprocessor to determine the presence, absence, or concentration of one or more analytes by determining a radiation signature of the electromagnetic radiation, and correlating the radiation signature of the electromagnetic radiation with information representative of the presence, absence, or concentration of one or more analytes.

9. The method of claim 7, wherein the sample analyte reader is an electrochemical reader, and using the sample analyte reader includes using the electrochemical reader to measure an electrical response from a biological sample on a disposable printed circuit board to measure the presence, absence, or concentration of one or more analytes in the biological sample.

10. The method of claim 7, wherein the sample analyte reader is an optical chemical strip reader, and using the sample analyte reader includes using the optical chemical strip reader to measure a change in color from a biological sample on a chemical test strip to measure the presence, absence, or concentration of one or more analytes in the biological sample.

11. The method of claim 7, wherein the non-invasive analyte reader and the sample analyte reader have separate respective microprocessors, and the method further includes isolating the functions of the non-invasive analyte reader and the sample analyte reader with the separate respective microprocessors for the non-invasive analyte reader and the sample analyte reader.

12. The method of claim 7, further including comparing one or more readings taken by the non-invasive analyte reader with one or more readings taken by the sample analyte reader to ensure that the one or more non-invasive measurement readings conform with the one or more sample analyte measurement readings.

13. A method of using a combination non-invasive analyte reading and sample analyte reading device, the combination non-invasive analyte reading and sample analyte reading device including a device housing, one or more outputs carried by the device housing, one or more inputs carried by the device housing to control operation of the device, a non-invasive analyte reader carried by the device housing, and a sample analyte reader carried by the device housing, comprising:
a) using the sample analyte reader of the combination non-invasive analyte reading and sample analyte reading device to take a sample analyte measurement reading of the presence, absence, or concentration of one or more analytes in a biological sample of a subject;
b) after step a, using the non-invasive analyte reader of the combination non-invasive analyte reading and sample analyte reading device to take a non-invasive measurement reading of the presence, absence, or concentration of one or more analytes in a tissue of a subject;
c) comparing the non-invasive measurement reading with the sample analyte measurement reading to ensure that the non-invasive measurement reading conforms with the sample analyte measurement reading; and
d) repeating steps b and c if the non-invasive measurement reading does not conform with the sample analyte measurement reading.

14. The method of claim 13, wherein the non-invasive analyte reader includes a radiation source, a radiation detector, and a microprocessor, and using the non-invasive analyte reader of the combination non-invasive analyte reading and sample analyte reading device to take a non-invasive measurement reading includes exposing at least a portion of the tissue of the subject to electromagnetic radiation from the radiation source; obtaining a measurement of electromagnetic radiation from the tissue using the radiation detector, and using the microprocessor to determine the presence, absence, or concentration of one or more analytes by determining a radiation signature of the electromagnetic radiation, and correlating the radiation signature of the electromagnetic radiation with information representative of the presence, absence, or concentration of one or more analytes.

15. The method of claim 13, wherein the sample analyte reader is an electrochemical reader, and using the sample analyte reader includes using the electrochemical reader to measure an electrical response from a biological sample on a disposable printed circuit board to measure the presence, absence, or concentration of one or more analytes in the biological sample.

16. The method of claim 13, wherein the sample analyte reader is an optical chemical strip reader, and using the sample analyte reader includes using the optical chemical strip reader to measure a change in color from a biological sample on a chemical test strip to measure the presence, absence, or concentration of one or more analytes in the biological sample.

17. The method of claim 13, wherein the non-invasive analyte reader and the sample analyte reader have separate respective microprocessors, and the method further includes isolating the functions of the non-invasive analyte reader and the sample analyte reader with the separate respective microprocessors for the non-invasive analyte reader and the sample analyte reader.

18. The method of claim 13, further including calibrating the non-invasive analyte reader based on one or more non-invasive measurement readings and one or more sample analyte measurement readings.
